(19) United States
(12) Patent Application Publication
(10) Pub. No.: US 2011/0086811 A1
(43) Pub. Date: Apr. 14, 2011

(54) HIGH-CONCENTRATION RUTIN-CONTAINING DRINK

(75) Inventors: Izumi AWANO, Makinohara-shi (JP); Naoya Hase, Makinohara-shi (JP); Masashige Taniguchi, Makinohara-shi (JP)

(73) Assignee: ITO EN, LTD.

(21) Appl. No.: 12/970,668

(22) Filed: Dec. 16, 2010

Related U.S. Application Data
(63) Continuation of application No. PCT/JP2009/063822, filed on Aug. 4, 2009.

(30) Foreign Application Priority Data
Aug. 5, 2008 (JP) 2008-201486

(51) Int. Cl. A61K 31/7048

(52) U.S. Cl. 514/27

(57) ABSTRACT

It is an object of the present invention to provide a packaged high-concentration rutin-containing drink suppressed the occurrence of the precipitation though it contains rutin in a high concentration. According to the invention, a packaged high-concentration rutin-containing drink includes rutin and quercetin is provided. The concentration of rutin is in a range from 70 to 1100 ppm and the ratio [(A)/(B)] by weight of (A) rutin to (B) quercetin is 3.7 to 58.0.

### CROSS REFERENCE TO RELATED APPLICATIONS

This is a Continuation Application of PCT Application No. PCT/JP2009/063822, filed Aug. 4, 2009, which was published under PCT Article 21(2) in Japanese.

This application is based upon and claims the benefit of priority from prior Japanese Patent Application No. 2008-201486, filed Aug. 5, 2008, the entire contents of which are incorporated herein by reference.

### BACKGROUND OF THE INVENTION

1. Field of the Invention

The present invention relates to a packaged drink containing rutin in a high concentration.

2. Description of the Related Art

Rutin (also referred to as rutoside or quercetin-3-rutinoside) is a glycoside in which rutinose is linked with quercetin. Rutin has physiological activities such as a capillary contracting action and action of reinforcing capillaries. Therefore, rutin is said to have an effect of preventing cerebral hemorrhage and the like. Further, rutin has antioxidizing ability. Accordingly, rutin has the function to remove active oxygen to thereby purify blood. Moreover, rutin is said to have such an anti-allergen function as to suppress the release of histamine.

Rutin is widespread in nature and is contained much in vegetables such as flower buds of a Japanese Pagoda tree in the family Fabaceae and buckwheat in the family Polygonaceae. Conventionally, buckwheat tea is among foods containing rutin. This buckwheat tea is highly healthy and tasty and therefore widely drunk (for example, Jpn. Pat. Appln. KOKAI Publication No. 60-262585). However, the amount of rutin contained in commercially available buckwheat tea which has been used so far is about 6 mg/100 g (60 ppm).

### BRIEF SUMMARY OF THE INVENTION

The inventors of the present invention have tried to produce a rutin enriched drink and, as a result, obtained a buckwheat extract having a high concentration. However, the problem that the precipitation is occurred in the buckwheat extract having a high concentration has been revealed. Because many transparent containers such as PET bottles are currently used as drink bottles, occurrence of the precipitate reduces product value. Therefore, the occurrence of the precipitation is undesirable. In light of this, it is an object of the present invention to provide a packaged high-concentration rutin-containing drink suppressed the occurrence of the precipitation though it contains rutin in a high concentration.

The inventors of the present invention have made earnest studies and as a result, found that quercetin derived from raw materials contained in drinks relates to the precipitation. Quercetin is a component contained in buckwheat. It is therefore considered that when the concentration of the buckwheat extract is raised to increase the concentration of rutin, the concentration of quercetin is increased with increase in the concentration of rutin, causing the precipitation. However, the inventors of the present invention have found that the precipitation is limited by adding rutin to the extract and by adjusting the ratio of rutin to quercetin contained in the extract to a certain range, even in the case where the concentration of the extract is high.

The present invention provides a packaged high-concentration rutin-containing drink comprising rutin and quercetin, wherein the concentration of rutin is in a range from 70 to 1100 ppm and the ratio [(A)/(B)] by weight of (A) rutin to (B) quercetin is 3.7 to 58.0.

Further, according to another aspect of the present invention, there is provided a method of producing a packaged high-concentration rutin-containing drink and a method of suppression of the precipitation by adjusting the concentration of rutin in the drink to a range from 70 to 1100 ppm and the ratio [(A)/(B)] by weight of (A) rutin to (B) quercetin in the drink to 3.7 to 58.0. Here, the above rutin is preferably α-glucosylated rutin.

The present invention can provide a high-concentration rutin-containing drink which is limited in the precipitation and has a good flavor and taste even though it contains rutin in a high concentration.

### DETAILED DESCRIPTION OF THE INVENTION

Hereinafter, the present invention will be explained in detail.

The packaged high-concentration rutin-containing drink of the invention comprises rutin as a component (A) and quercetin as a component (B), wherein the ratio [(A)/(B)] by weight of the component (A) to the component (B) is 3.7 to 58.0.

The above concentration of rutin and ratio by weight of rutin/quercetin are attained by adding water-soluble rutin in an extract obtained by extracting the raw materials. In other words, the concentration of rutin in the drink of the present invention means the concentration of the sum of natural rutin derived from the raw materials and the added water-soluble rutin.

Alternatively, the above concentration of rutin and ratio by weight of rutin to quercetin can be attained by restraining the phenomenon that rutin is decomposed into quercetin. Specifically, in the extract which is obtained by extracting the raw material having a high concentration and contains rutin in a high concentration, a rutin decomposition enzyme derived from the raw materials is deactivated to thereby make it possible to attain the above objects. The deactivation of the rutin decomposition enzyme may be accomplished by wet heat treatment of the raw materials as disclosed in Japanese Patent No. 2766259. Further, the above concentration of rutin and ratio by weight of rutin to quercetin can be attained by removing quercetin in the raw material extract containing the rutin in a high concentration by a known filtrating method.

Here, the term “water-soluble rutin” means rutin so modified that its solubility in water is increased. As water-soluble rutin, α-glucosylated rutin is preferably used. This α-glucosylated rutin means a compound obtained by α-glucosylating of rutin through the transition action of an enzyme such as glucose. Examples of water-soluble rutin include monoglucosyl rutin, diglucosyl rutin, triglucosyl rutin and tetraglucosyl rutin, monoglucosyl rutin being preferable. As the commercially available product of the water-soluble rutin (α-glucosylated rutin), for example, a product available from Toyo Sugar Refining Co., Ltd. under the name of “αG rutin PS-D” may be used. This α-glucosylated rutin is also called “enzyme treated rutin”.

The “quercetin” is contained much in, for example, capers, apples, teas (for example, tea plant), onions, grapes, broccolis, mulukhiya, raspberries, cowberries, cranberries, opuntias, leaf vegetables, citrus and honeys and is also contained in buckwheat. Quercetin to be contained in the drink of the present invention is derived from raw materials.

The description that rutin is contained in a high concentration in the drink of the present invention means that rutin is contained in a concentration higher than that contained originally in the extract which is constitute the drink. For example, buckwheat tea is intended to contain about 70 ppm or more of rutin, the concentration of rutin is not limited to this but differs depending on the type of drink.

Further, in the drink of the present invention, the content of rutin is preferably about 1100 ppm or less. When the content of rutin exceeds about 1100 ppm, the drink is bitterer and has an unfavorable flavor and taste, showing that it is unsuitable as a drink. The content of rutin in the drink of the present invention is more preferably in a range from about 80 to 1050 ppm, even more preferably about in a range from about 90 to 1000 ppm and even more preferably in a range from about 100 to 840 ppm.

In the drink of the present invention, the ratio by weight of rutin/quercetin is preferably about 3.7 to 58.0. When the ratio by weight of rutin/quercetin is less than 3.7, there is a fear that the precipitation in the packaged drink cannot be suppressed efficiently. When the ratio by weight of rutin/quercetin exceeds 58.0 on the other hand, the concentration of rutin is so high that the drink is bitterer and has an unfavorable flavor and taste, showing that it lacks in the qualities required for a drink. The ratio by weight of rutin/quercetin is preferably in a range from about 3.7 to 58.0, more preferably in a range from about 6.3 to 55.0, even more preferably in a range from about 9.5 to 52.0 and even more preferably in a range from about 32.2 to 46.7.

Although no particular limitation is imposed on the content of quercetin in the drink of the present invention, the content of quercetin is in a range from 1.2 to 297.3 ppm when it is calculated from the content of rutin and the ratio by content of rutin/quercetin. It is considered that precipitates tend to be occurred more easily with increase in the content of quercetin.

The term “drink” in the invention means drinking products obtained by processing and adjusting an extract obtained by extracting or pressing juice out of vegetables used as raw materials.

The raw materials to be used in the present invention are preferably vegetables containing rutin in nature. The raw materials are preferably, though not limited to, vegetables having a high rutin content such as seeds or all grasses of buckwheat, tea leaves produced from the leaves and stems of tea trees (Camellia sinensis var. sinensis, Camellia sinensis var. assamica, or their hybrids), for example, non-fermented teas such as a green tea of medium quality, green tea of the highest quality, covered tea (Kabusecha), coarse tea and pan-fried tea (Kamairicha), weakly-fermented teas such as a flowering tea and white tea obtained by clinging the flavor of flowers to, for example, jasmine tea, semi-fermented teas such as Oolong tea and fermented teas such as a black tea, or citrus. Particularly, tartary buckwheat is preferable.

Furthermore, honey, adzuki beans, tomatoes, figs, cherries, apricots, asparaguses, potatoes and the like also contain rutin and may be therefore used as the raw materials.

Besides the above, the leaves and stems of various vegetable such as unpolished rice, barley, wheat, tear grass, corn, amaranth, quinoa, Nanban kiwi (millet), Nemacysis decipiens, Glycyrrhiza, lotus, shiso, pine tree, plantain, rosemary, mulberry tree, Gymnema, Cassia obtusifolia Linn, soybean, kelp, Ganoderma lucidum (Leyss.ex.Fr.) Karst, Sasa veitchii, persimmon, sesame, safflower, Angelica keiskei, Chinpi (fruit skins of ripe Citrus reticulate), Guava, aloe, Gymnema, Eucommia ulmoides, Houttuynia cordata, chicory, evening primrose and loquat may be combined.

In the present invention, the “packaged drink” means, though not limited to, drinks filled or sealed in a container. The container can be made of a material selected from a metal, glass, plastic and paper or paper materials composed of a metal or plastic film, and a combination of these materials.

When a transparent bottle such as a glass bottle, a molded container using polyethylene terephthalate as its major component (so-called PET bottle) and a plastic bottle including a multilayer molded container provided with a barrier layer such as an oxygen barrier layer is used, a drink filled in the container can be visually observed from outside. It is therefore important to suppress or prevent the precipitation in the drink.

Next, a method of producing the packaged high-concentration rutin-containing drink of the present invention will be explained. The packaged high-concentration rutin-containing drink of the present invention may be produced by obtaining an extract from vegetables which are raw materials and by adding water-soluble rutin. Preferably, after the concentration of rutin derived from the raw materials is raised by increasing the concentration of the extract, water-soluble rutin is further added to produce a high-concentration rutin-containing drink. However, the method is not limited to this, and a high-concentration rutin-containing drink may be produced by adding water-soluble rutin to an extract having a usual concentration. A person skilled in the art can optionally select the concentration of the extract in consideration of various factors such as the type of raw material, production process and the type of final drink.

First, the raw materials are extracted or squeezed. The condition of the extraction is properly selected depending on factors such as the type of raw material, type of extraction machine and form of a final product. The extraction may be performed by dipping the raw materials in hot water or by spraying hot water on the raw materials. On the other hand, the squeezing of the raw materials is performed by crushing the raw material and by squeezing or straining the crushed materials. A squeezed juice may be one obtained by concentrating the above juice or one obtained by diluting a concentrated juice to return it to a juice form.

Although a method of preparing a drink by using the extract obtained by extracting the raw materials will be explained, a drink can be also prepared in the same manner by using the juice.

The extract obtained in the above manner is cooled and is subjected to solid-liquid separation performed by a filtering method, for example, using a cartridge filter, flannel filter cloth, filter plate, or filter paper, or filtering method such as a filter press using a filtering adjuvant together or by centrifugation method. The raw material particles and tea leaves are removed from the extract by this process.

Optionally, a known method for limiting or preventing the precipitation may be carried out. The method include for example, a method of decomposing water-soluble polysaccharide components by oxygen treatment and a method of physically removing causal materials or precipitates by ultrafiltration or filtration using diatomaceous earth. Though these methods do not heighten the effect of suppression of the precipitation in the packaged drink of the present invention, they may be carried out, for example to restrain the turbidity (i.e. primary sediments) just after the drink is filled.

Then, water-soluble rutin is added to the extract such that the concentration of rutin in the drink is in a range from 70 to 1100 ppm and the ratio [(A)/(B)] by weight of (A) rutin to (B) quercetin is in a range from 3.7 to 58.0 to make a preparation solution. The preparation solution may be made in a concentration adequate to be able to provide the solution as a drink or may be a concentrated solution which is to be further diluted to prepare a final drink. The amount of rutin to be added may be properly adjusted according to the case.

The pH of the above preparation solution is adjusted according to the need by using Vitamin C and sodium ascorbate or a pH regulator such as sodium hydrogen carbonate, sodium bicarbonate and potassium carbonate. Further, according to the need, additives selected from antioxidants such as ascorbic acid and sodium ascorbate, perfumes, emulsifiers, preservatives, sweeteners, coloring materials, thickening stabilizers, seasonings and fortifiers may be formulated either singly or in combinations of two or more.

The packaged drink of the present invention may be sterilized in any one of the stages during the course of the process according to the need. As to the sterilizing method and the condition, it is only required to select a method and a condition producing the same effect as in the case of using the condition prescribed in the food hygiene law. In the case of using, as the container, a heat-resistant one, retort sterilization may be adopted. In the case of using a container having no heat resistance, for example, the preparation solution may be subjected to high-temperature and short-time sterilization using a plate type heat exchanger, then cooled to a predetermined temperature and then, hot-packed or aseptically filled after cooled.

After the drink is filled, each concentration of rutin and quercetin in the drink is measured. The concentrations of rutin and quercetin can be measured by quantitative analysis using a HPLC.

The packaged high-concentration rutin-containing drink produced in the above method according to the present invention is a drink which is suppressed in the precipitation though rutin is contained in a high concentration. Such a drink is advantageous when it is filled particularly in a transparent container.

Moreover, according to another aspect of the present invention, there is provided a method of suppression of the precipitation in a packaged high-concentration rutin-containing drink, the method comprising adding water-soluble rutin such that the concentration of rutin in the drink is in a range from 70 to 1100 ppm and adjusting the ratio [(A)/(B)] by weight of (A) rutin to (B) quercetin in the drink to 3.7 to 58.0.

### EXAMPLES

Hereinafter, the present invention will be explained in more detail by way of Examples, which are not intended to be limiting of the present invention.

In buckwheat tea, the concentration of rutin and the ratio by weight of rutin to quercetin were varied to observe whether or not precipitates were occurred.

In the tests 1 to 16 shown in Table 1, seeds of tartary buckwheat (Fagopyrum tataricum) from which hulls were removed were dipped in 95 to 98° C. hot water having a volume ten times that of the seeds for 90 minutes to obtain an extract. The extract was cooled to 25° C. or less. The cooled extract was filtered at 9200 rpm at a rate of 6 L/min by an centrifugal separator (trade name: SA1, manufactured by Westfalia Separator K.K.). Thereafter, the filtered extract was further filtered by using a CUNO Polyprocreen 1μ cartridge (trade name, manufactured by Sumitomo 3M Limited). The amount of tartary buckwheat used in each test example is as shown in Table 1.

In each test example excluding the test 3, water-soluble rutin (αG Rutin PS-D, manufactured by Toyo Sugar Refining Co., Ltd.) was added to the extract. The amount of water-soluble rutin to be added in each test example is as shown in Table 1. The above αG Rutin PS-D was dissolved in water having a volume about 50 times that of the αG Rutin PS-D prior to addition. In succession, 300 ppm of Vitamin C was added to each test example, sodium bicarbonate was added to adjust the solution after being sterilized to pH 5.7, 6.0 or 6.3 and quantitatively measured to prepare a preparation solution.

This preparation solution was sterilized by heating at 140° C. for 60 seconds. Then, the solution was cooled to 25° C. and aseptically filled in a PET bottle. Each concentration of rutin and quercetin in the obtained drink after the drink was filled was measured. The measurement was made in the following condition.

### 1. Sample

Authentic product of rutin: “Rutin”, manufactured by Wako Pure Chemical Industries, Ltd.

Authentic product of quercetin: “Quercetin”, manufactured by NACALAI TESQUE K.K.

Water-soluble rutin: “αG Rutin PS-D”, manufactured by Toyo Sugar Refining Co., Ltd.

### 2. Preparation of a Test Solution

Standard solution of rutin

“Rutin” manufactured by Wako Pure Chemical Industries, Ltd. was taken in a measuring flask and dissolved in 99% methanol to prepare a standard rutin solution having an appropriate concentration.

Standard solution of quercetin

“Quercetin” manufactured by NACALAI TESQUE K.K. was taken in a measuring flask and dissolved in 99% methanol to prepare a standard quercetin solution having an appropriate concentration.

Water-soluble rutin solution

“αG Rutin PS-D”, manufactured by Toyo Sugar Refining Co., Ltd. was taken in a measuring flask and dissolved in 99% methanol to prepare a standard rutin solution having an appropriate concentration.

Sample

Each drink of the tests 1 to 16 was diluted with 99% methanol to make the drink have a proper concentration, and then filtered using a 0.45 μm membrane filter to prepare a sample.

### 3. Condition of HPLC

HPLC device: AQUITY UPLC, manufactured by Waters Corporation

Column: C18 2.1×100 mm

Column temperature: 40° C.

Solvent: Solution A; acetonitrile:water:formic acid=5:95:0.1

Solvent: Solution B; acetonitrile:water:formic acid=95:5:0.1

Gradient: 0-2 min A:B=90:10→60:40

Gradient: 2-15 min A:B=90:10→60:40

Flow rate: 200 μl/min

Detecting wavelength: 354 nm

In this case, the sample was measured twice to find an average as the content.

### 4. Method of Calculation

(1) Rutin

Concentration of rutin (ppm) in sample=Concentration of standard solution×(Sum of peak areas of monoglucosyl rutin and rutin in sample/Peak area of rutin in standard solution)

(2) Quercetin

Concentration of quercetin (ppm) in sample=Concentration of standard solution×(Peak area of quercetin in sample/Peak area of quercetin in standard solution)

The concentration of rutin in the sample was calculated from the sum of the areas of monoglucosyl rutin and rutin in the sample, the area and concentration of rutin in the standard solution.

The content of rutin and quercetin in the authentic product was set to 100 to calculate.

A water-soluble rutin solution was used to confirm the peak of monoglucosyl rutin.

The concentrations of rutin and quercetin in each test example are shown in Table 1. In this case, the concentration of rutin shown in Table 1 is one measured after the water-soluble rutin is added. Specifically, this is the concentration of rutin including the rutin derived from the raw materials and water-soluble rutin added in the test example.

The drink after filled was allowed to stand to observe whether precipitates were occurred or not. Further, the flavor of the drink was examined. The results are shown in Table 1.

TABLE 1

| | Seeds of | Amount of rutin | Concentration | Concentration | | | | |
|---|---|---|---|---|---|---|---|---|
| | buckwheat | being added | of rutin | of quercetin | Rutin/quercetin | | | pH after |
| | g/L | g/L | ppm | ppm | ratio | Precipitation | Flavor | sterilization |
| Test 1 | 9 | 0.04 | 63 | 13 | 4.85 | − | ◯ | 6.3 |
| Test 2 | 12 | 0.04 | 76 | 17 | 4.47 | − | ◯ | 6.3 |
| Test 3 | 24 | — | 113 | 35 | 3.23 | + | ◯ | 6.3 |
| Test 4 | 12 | 0.1 | 108 | 17 | 6.35 | − | ◯ | 6.3 |
| Test 5 | 24 | 0.04 | 134 | 35 | 3.83 | − | ◯ | 6.3 |
| Test 6 | 24 | 0.1 | 168 | 35 | 4.80 | − | ◯ | 6.3 |
| Test 7 | 12 | 0.2 | 160 | 17 | 9.41 | − | ◯ | 6.3 |
| Test 8 | 12 | 0.2 | 160 | 17 | 9.41 | − | ◯ | 6.0 |
| Test 9 | 12 | 0.2 | 160 | 17 | 9.41 | − | ◯ | 5.7 |
| Test 10 | 35 | 0.04 | 183 | 50 | 3.66 | + | ◯ | 6.3 |
| Test 11 | 24 | 0.2 | 218 | 35 | 6.23 | − | ◯ | 6.3 |
| Test 12 | 12 | 1.0 | 579 | 18 | 32.17 | − | ◯ | 6.3 |
| Test 13 | 12 | 1.3 | 737 | 18 | 40.94 | − | ◯ | 6.3 |
| Test 14 | 12 | 1.5 | 841 | 18 | 46.72 | − | ◯ | 6.3 |
| Test 15 | 12 | 1.9 | 1051 | 18 | 58.39 | − | Δ | 6.3 |
| Test 16 | 12 | 2.0 | 1211 | 18 | 67.28 | − | X | 6.3 |

+: Precipitates are occurred,
−: No precipitate is occurred,
◯: Good flavor,
Δ: Slightly bad flavor,
X: Bad flavor

The tests 3 and 10 in which the precipitation is observed have rutin/quercetin ratios of 3.22 and 3.64 respectively. This shows that if the rutin/quercetin ratio is about 3.7 or more, there is a tendency that no precipitate is occurred. Further, though each of the tests 3, 5, 6 and 11 has almost the same quercetin concentration, in the test 3 to which no rutin is added and in which the rutin/quercetin ratio was 3.22, the precipitation was occurred. On the other hand, in the tests 5, 6 and 11 in which the rutin/quercetin ratios were 3.82, 4.79 and 6.20, no precipitate is occurred. It is also shown from this fact that the precipitation can be suppressed by adding rutin and by controlling the rutin/quercetin ratio to a predetermined value or more.

On the other hand, the tests 15 and 16 have rutin/quercetin ratios of 58.06 and 66.52 respectively. There is a tendency that these drinks each have a bitter taste and unacceptable flavor though no precipitate is occurred. Accordingly, from the viewpoint of commercial products, it is shown to be desirable that the rutin/quercetin ratio does not exceed 58.0. Moreover, it is shown from Table 1 that the appropriate concentration of rutin in the drink is in a range from about 70 to 1100 ppm.

From the above results, there is a tendency that the stability is impaired in the case where the amount of rutin is smaller than that of quercetin. It is supposed that there is the possibility that quercetin molecules are combined among them into an aggregated material, causing the precipitation and water soluble rutin penetrates between quercetins, thereby preventing the production of coagulates of quercetin and precipitates.

As is described above, the present invention can provide a high-concentration rutin-containing drink suppressed in the precipitation by controlling the concentration of rutin and the ratio of rutin/quercetin. The present invention can be advantageously applied to drinks using vegetables containing rutin and quercetin as raw materials in the production of these drinks.

What is claimed is:

1. A packaged high-concentration rutin-containing drink comprising rutin and quercetin, wherein the concentration of rutin is in a range from 70 to 1100 ppm and the ratio [(A)/(B)] by weight of (A) rutin to (B) quercetin is 3.7 to 58.0.

2. The drink according to claim 1, comprising an extract of buckwheat, an extract of tea or juices of citrus.

3. The drink according to claim 2, which is buckwheat tea.

4. A method of producing a packaged high-concentration rutin-containing drink, the method comprising adjusting the concentration of rutin in the drink to a range from 70 to 1100 ppm and the ratio [(A)/(B)] by weight of (A) rutin to (B) quercetin in the drink to 3.7 to 58.0.

5. The production method according to claim 4, wherein the rutin is α-glucosylated rutin.

6. The production method according to claim 5, wherein the drink is prepared from an extract of buckwheat, an extract of tea or juices of citrus.

7. The production method according to claim 6, wherein the drink is prepared from an extract of buckwheat.

8. A method of suppression of the precipitation in a packaged high-concentration rutin-containing drink, comprising adjusting the concentration of rutin in the drink to a range from 70 to 1100 ppm and the ratio [(A)/(B)] by weight of (A) rutin to (B) quercetin in the drink to 3.7 to 58.0.
